(19) United States
(12) Patent Application Publication
(10) Pub. No.: US 2012/0089033 A1
(43) Pub. Date: Apr. 12, 2012

(54) BIOINFORMATION DETECTING DEVICE

(76) Inventor: Tetsu Nemoto, Ishikawa (JP)

(21) Appl. No.: 13/265,675

(22) PCT Filed: Apr. 21, 2010
(86) PCT No.: PCT/JP2010/057062

§ 371 Date: Dec. 21, 2011

(30) Foreign Application Priority Data
Apr. 23, 2009 (JP) 2009-104599

(51) Int. Cl. A61B 5/113; A61B 5/0205

(52) U.S. Cl. 600/484

(57) ABSTRACT

A bioinformation detecting device 2 includes a pressure sensitive plate 10, a substrate 30, a clamping force variation detecting mechanism 50 arranged between the pressure sensitive plate 10 and the substrate 30, and a signal combining unit 200. The clamping force variation detecting mechanism 50 includes a first piezoelectric element 60, a transmission member 70, and a second piezoelectric element 80 in this order. If a clamping force varies in response to vibration acting on a pressure sensitive surface, the transmission member transmits the variation to both the first and second piezoelectric elements 60 and 80 to make the first and second piezoelectric elements 60 and 80 generate signals. The signal combining unit 200 combines the first and second detected signals to detect bioinformation. This allows reduction of a measurement burden placed on an organism.

### TECHNICAL FIELD

The present invention relates to a bioinformation detecting device for detecting bioinformation such as a heart rate, body movement, and a body posture.

### BACKGROUND ART

Patent Literature 1 (Japanese Patent Application Laid-Open No. 2007-54606) suggests a conventional bioinformation detecting device for detecting bioinformation such as the breathing and body movement of an organism based on vibration generated by the breathing or body movement of the organism. The invention disclosed in Patent Literature 1 includes a base provided with an organism holding part, a flexible pressure transmitting part, and a sensor part for detecting pressure change. Further, a space is formed between the organism holding part and the base, or between the pressure transmitting part and the base.

Patent Literature 2 (Publication of International Application No. WO 2007/029326) suggests a detecting device for detecting the heart rate, breathing and the like of a small animal. The invention disclosed in Patent Literature 2 also includes a base provided with a vibration transmitting plate of great flexibility. Further, a piezoelectric transducer which is capable of detecting an acceleration and the like is stuck to the vibration transmitting plate, thereby directly detecting the vibration of the vibration transmitting plate. In order to detect the minute vibration of a small animal, the thickness of the vibration transmitting plate should be reduced to enhance sensitivity. However, this in turn reduces the strength of the vibration transmitting plate. In view of this, Patent Literature 2 also discloses a technique of enhancing load bearing without involving vibration attenuation by providing a spacer of high elasticity at the center of the vibration transmitting plate.

### SUMMARY OF INVENTION

### Problems to be Solved by Invention

The invention disclosed in Patent Literature 1 detects pressure change generated by the breathing and the like of an organism on the basis of the distortion of the flexible pressure transmitting part. Accordingly, a space should be formed between the organism holding part and the base, or between the pressure transmitting part and the base on which the pressure transmitting part is provided. This imposes limitations on the thickness reduction of the device, placing a heavy measurement burden on a subject to be measured.

The invention of Patent Literature 2 discloses a technique of directly detecting the vibration of the vibration transmitting plate with a sensor. This technique however requires the thinning of the vibration transmitting plate to vibrate easily in order to enhance sensitivity. Accordingly, load bearing should be enhanced by using a spacer made of an elastic member in the form as a spindle. This results in a problem in that the technique of patent literature 2 cannot be applied to relatively heavy organisms such as humans, dogs, and cats.

In view of the aforementioned current conditions, the present invention is intended to provide a bioinformation detecting device capable of reducing a measurement burden to be placed on a subject to be measured.

### Means for Solving Problems

The present invention to achieve the aforementioned object is intended for a bioinformation detecting device for detecting bioinformation such as the breathing condition, heartbeat, and body movement of an organism based on vibration generated by the breathing motion of the organism. The bioinformation detecting device includes: a pressure sensitive plate with a pressure sensitive surface for detecting the vibration; a substrate arranged to face the pressure sensitive plate; a clamping force variation detecting mechanism arranged between the pressure sensitive plate and the substrate, the clamping force variation detecting mechanism detecting variation in a clamping force applied between the pressure sensitive plate and the substrate based on the vibration acting on the pressure sensitive surface; and a signal combining unit for combining a plurality of detected signals output from the clamping force variation detecting mechanism. The clamping force variation detecting mechanism includes: a first piezoelectric element arranged on the pressure sensitive plate on the opposite side of the pressure sensitive surface, the first piezoelectric element converting the variation in a clamping force acting on the first piezoelectric element itself to a first detected signal; a transmission member arranged on the first piezoelectric element on the opposite side of the pressure sensitive plate, the transmission member transmitting the variation in a clamping force; and a second piezoelectric element arranged between the transmission member and the substrate, the second piezoelectric element converting the variation in a clamping force acting on the second piezoelectric element itself to a second detected signal. The variation in a clamping force generated based on the vibration acting on the pressure sensitive surface is transmitted through the transmission member to both the first and second piezoelectric elements, and is converted to the first and second detected signals. The first and second detected signals are combined by the signal combining unit, thereby detecting the bioinformation.

In the bioinformation detecting device, the present invention to achieve the aforementioned object is characterized in that a support member for maintaining a constant distance between the pressure sensitive plate and the substrate is arranged between the pressure sensitive plate and the substrate in a region surrounding the clamping force variation detecting mechanism.

In the bioinformation detecting device, the present invention to achieve the aforementioned object is characterized in that a pre-load is applied to the clamping force variation detecting mechanism in a stationary condition by setting a distance between the pressure sensitive plate and the substrate maintained by the support member to be smaller than the size of the clamping force variation detecting mechanism in the direction of the variation in a clamping force determined while no load is applied to the clamping force variation detecting mechanism.

In the bioinformation detecting device, the present invention to achieve the aforementioned object is characterized by further including a signal extracting unit for extracting a heartbeat component by filtering an output signal from the signal combining unit.

In the bioinformation detecting device, the present invention to achieve the aforementioned object is characterized in that the support member is provided at least in each of the four corners of the pressure sensitive plate, and that the clamping force variation detecting mechanism is disposed at a position that is substantially the center of the pressure sensitive plate.

Preferably, the present invention further includes an analyzing unit for analyzing an output signal from the signal combining unit, and an output signal from the signal extracting unit. Preferably, the analyzing unit smoothes the signals further and detects respective peak values of the waveforms by separating at least the breathing condition, heartbeat, and body movement of a human body simultaneously or independently. The analyzing unit also allows determination of the posture of a human body including rolling over during sleep. Still preferably, the analyzing unit includes a frequency separating means for separating variation in a detected voltage to detect bioinformation.

Preferably, the transmission member of the present invention has a transmission surface smaller in area than the pressure receiving surfaces of the first and second piezoelectric elements.

Preferably, the present invention includes a plurality of detecting modules each with a pressure sensitive plate, a substrate, a clamping force variation detecting mechanism, and others, and these detecting modules simultaneously make measurements at a plurality of sites for measurement of an organism to detect the breathing condition, heartbeat, and body movement of the organism. The sites for measurement of an organism preferably include at least a head region and an abdominal region, and preferably, also include a foot region.

### Advantageous Effects of Invention

The present invention achieves an excellent effect as the invention is capable of precisely detecting bioinformation such as breathing, body movement, and a body posture.

### BRIEF DESCRIPTION OF DRAWINGS

FIG. 1 is a diagram showing the entire structure of a bioinformation detecting device according to an embodiment of the present invention.

FIG. 2(a) is a plan view of a detecting module of the bioinformation detecting device, and FIG. 2(b) is a front view of the detecting module.

FIG. 3 is a block diagram showing a circuit structure in the bioinformation detecting device.

FIG. 4 includes graphs (A) and (B) relating to Example and Comparative Example, respectively, which show waveforms formed when a breathing component is detected in a signal obtained under a head region by the bioinformation detecting device.

FIG. 5 includes graphs (A) and (B) relating to Example and Comparative Example, respectively, which show waveforms formed when a breathing component is detected in a signal obtained under an abdominal region by the bioinformation detecting device.

FIG. 6 includes graphs (A) and (B) relating to Example and Comparative Example, respectively, which show waveforms formed when a heartbeat component is detected in a signal obtained under a head region by the bioinformation detecting device.

FIG. 7 includes graphs (A) and (B) relating to Example and Comparative Example, respectively, which show waveforms formed when a heartbeat component is detected in a signal obtained under an abdominal region by the bioinformation detecting device.

### EMBODIMENT(S) FOR CARRYING OUT INVENTION

An embodiment of the present invention will be described next with reference to the accompanying drawings.

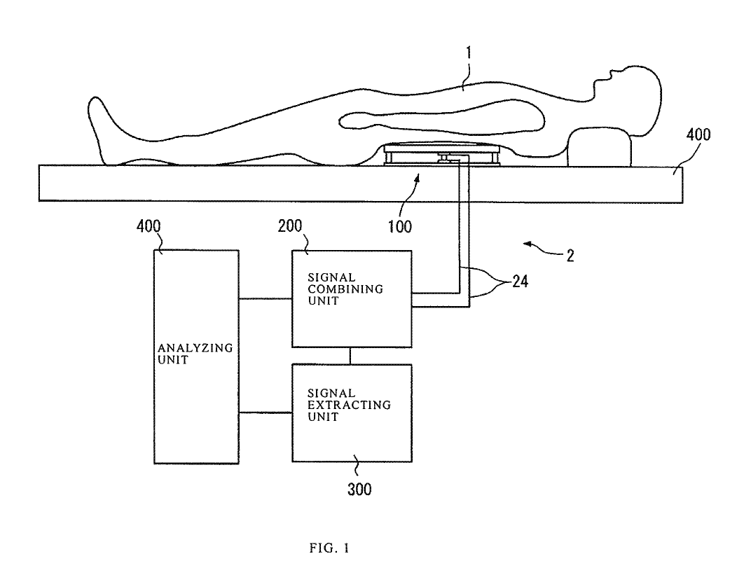

FIG. 1 shows the entire structure of a bioinformation detecting device 2 according to the embodiment of the present invention. The bioinformation detecting device 2 includes a detecting module 100, a signal combining unit 200, a signal extracting unit 300, and a signal analyzing unit 400. The detecting module 100 detects the bioinformation of an organism 1 such as breathing condition, heartbeat, and body movement based on vibration generated by the breathing motion of the organism 1. The signal combining unit 200 combines a plurality of detected signals output from the detecting module 100. The signal extracting unit 300 filters an output signal from the signal combining unit 200 to extract a heartbeat component. The signal analyzing unit 400 uses both the signals from the signal combining unit 200 and the signal extracting unit 300 to perform noise removal and the like of the output signals, and analyzes the breathing, heartbeat, body movement, and body posture of the organism 1 based on a result of the noise removal.

The detecting module 100 is placed under each of the abdominal region and the head region of the organism 1 if the organism 1 lies on a bed 500. In order to reduce a measurement burden to be placed on the organism 1, it is preferable that the detecting module 100 be placed under a pillow (for measurement of a head region, for example), and under a mattress (for measurement of an abdominal region or a foot region, for example). The detecting module 100 is provided with an output cable 24 through which detected signals are transmitted to the unit.

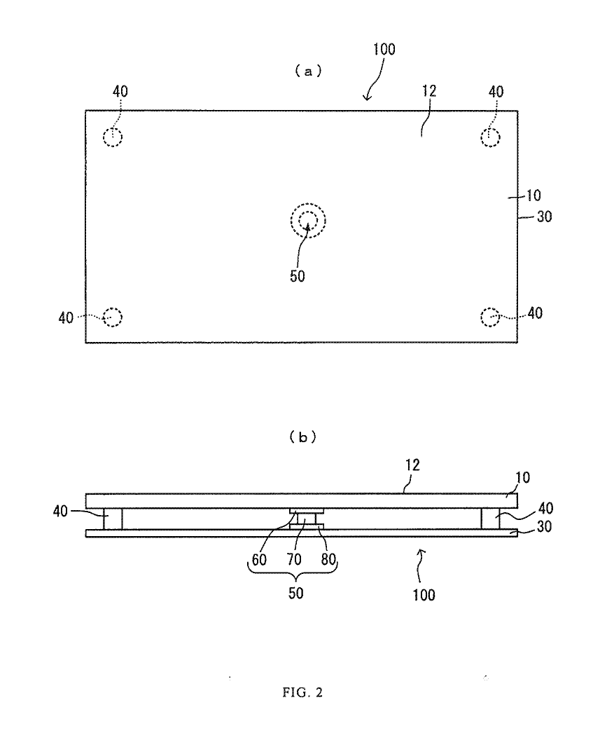

FIG. 2 shows the detecting module 100 in an enlarged manner. FIGS. 2(a) and 2(b) are plan and front views, respectively, of the bioinformation detecting device 2. The detecting module 100 includes a pressure sensitive plate 10, a substrate 30, support members 40, and a clamping force variation detecting mechanism 50.

The pressure sensitive plate 10 is a thin plate member (in the form of a plate) made of a flexible resin or a flexible metal. The pressure sensitive plate 10 has a pressure sensitive surface 12 that detects vibration transmitted from an organism. The material of the pressure sensitive plate 10 is not limited to a resin or a metal. The pressure sensitive plate 10 may be made of a different material such as ceramics preferably having high rigidity that will not generate delay in signal transmission. The pressure sensitive plate 10 of the present embodiment is a rectangular thin plate member (in the form of a plate). However, the shape of the pressure sensitive plate 10 is not limited thereto, but the pressure sensitive plate 10 may also be a square or circular thin plate member (this also applies to the substrate 30 described later). The area of the pressure sensitive surface 12 is set to be greater than that of a pressure receiving surface of the clamping force variation detecting mechanism 50 described later. The pressure sensitive plate 10 has rigidity and mechanical strength that allow the pressure sensitive plate 10 to stand the weight of an organism. Accordingly, even if the pressure sensitive plate 10 receives a weight (load) from a relatively heavy organism such as a human, a dog and a cat, the pressure sensitive plate 10 deforms an amount not exceeding an amount that cannot be detected by the clamping force variation detecting mechanism 50. The present embodiment uses a plastic plate of a thickness of 5 mm as the pressure sensitive plate 10. The size of the pressure sensitive plate 10 is 60 cm×20 cm, for example.

The substrate 30 is arranged to face the pressure sensitive plate 10, and is composed of a plate member (in the form of a plate) made of a resin or a metal having high rigidity. The material of the substrate 30 is not limited to such a resin or metal. The substrate 30 may be made of a different material such as ceramics preferably having high rigidity that will not generate delay in signal transmission. The substrate 30 of the present embodiment is a rectangular plate member. However, the shape of the substrate 30 is not limited thereto, but the substrate 30 may also be a square or circular plate member (in the form of a plate). The substrate 30 is placed on a floor surface and the like, so that the rigidity of the substrate 30 can be maintained by the integration of the substrate 30 with the floor surface. This allows the substrate 30 to be thinner than the pressure sensitive plate 10 that maintains its rigidity only by the pressure sensitive plate 10 itself, thereby realizing thickness reduction of the entire detecting module 100. The present embodiment uses a plastic plate of a thickness of 3 mm.

The support members 40 are metallic columnar members of high rigidity. Four support members 40 are placed in the respective four corners between the pressure sensitive plate 10 and the substrate 30. The support members 40 have upper and lower surfaces fixed to the pressure sensitive plate 10 and the substrate 30 to maintain a constant distance between the pressure sensitive plate 10 and the substrate 30. In the present embodiment, a distance between the pressure sensitive plate 10 and the substrate 30 (length of the support members 40) is set to 5 mm. The material of the support members 40 is not limited to a metal of high rigidity. The support members 40 may be made of a different material such as a high-rigid resin and ceramics preferably having high rigidity that will not generate delay in signal transmission. Further, the support members 40 do not necessarily have a columnar shape, but may also have a shape that is preferably a square pole or a triangle pole.

The clamping force variation detecting mechanism 50 is arranged between the pressure sensitive plate 10 and the substrate 30. Based on vibration acting on the pressure sensitive surface 12, the clamping force variation detecting mechanism 50 detects variation in a clamping force applied between the pressure sensitive plate 10 and the substrate 30. The clamping force variation detecting mechanism 50 includes a first piezoelectric element 60, a transmission member 70, and a second piezoelectric element 80 placed in this order in a direction from the pressure sensitive plate 10 toward the substrate 30. The opposite ends of the support members 40 are fixed to the pressure sensitive plate 10 and the substrate 30 respectively in a region surrounding the clamping force variation detecting mechanism 50, thereby maintaining a constant distance between the pressure sensitive plate 10 and the substrate 30 as described above.

The first piezoelectric element 60 is arranged on the pressure sensitive plate 10 on the opposite side of the pressure sensitive surface 12. The first piezoelectric element 60 converts variation in a clamping force acting on the first piezoelectric element 60 itself to a first detected signal. The first piezoelectric element 60 has a circular plate shape. In response to variation in a clamping force applied to the first piezoelectric element 60, the first piezoelectric element 60 generates voltages at its opposite ends responsive to the applied pressure (a negative voltage at one end and a positive voltage at the opposite end). This first piezoelectric element is not necessarily in the shape of a circular plate, but may be in a different shape that is preferably a rectangle or a square, for example. The first piezoelectric element 60 is fixed to the pressure sensitive plate 10 with an adhesive agent made of a silicone resin. The adhesive agent made of a silicone resin exhibits excellence in electric insulation, water resistance, and heat resistance.

The transmission member 70 is a columnar member made of a high-rigid metal. The transmission member 70 is arranged on the first piezoelectric element 60 on the opposite side of the pressure sensitive plate 10, and transmits variation in a clamping force. The first piezoelectric element 60 and the transmission member 70 are fixed with an elastic adhesive agent made of an epoxy resin. The material of the transmission member 70 is not limited to a high-rigid metal. The transmission member 70 may be made of a different material such as a high-rigid resin and ceramics preferably having high rigidity that will not generate delay in signal transmission.

The second piezoelectric element 80 has the same structure as that of the first piezoelectric element 60. The second piezoelectric element 80 is arranged on the transmission member 70 on the opposite side of the first piezoelectric element 60, namely arranged between the transmission member 70 and the substrate 30. The second piezoelectric element 80 converts variation in a clamping force acting on the second piezoelectric element 80 itself to a second detected signal. The second piezoelectric element 80 and the transmission member 70 are fixed (tightly connected) with an elastic adhesive agent made of an epoxy resin. The second piezoelectric element 80 and the substrate 30 are fixed (tightly connected) with an adhesive agent made of a silicone resin. To be specific, the second piezoelectric element 80 is provided between the transmission member 70 and the substrate 30 integrally with the transmission member 70 and the substrate 30 (such that substantially no space is left between the second piezoelectric element 80 and the transmission member 70, and between the second piezoelectric element 80 and the substrate 30). Thus, the second piezoelectric element 80 is capable of converting pressure (variation in a clamping force) received from the transmission member 70 to a voltage (second detected signal) without loss.

The first and second piezoelectric elements 60 and 80 are elements that generate voltages by the action of piezoelectric effect (piezoelectric phenomenon) responsive to pressure (clamping force) applied in a thickness direction. The first and second piezoelectric elements 60 and 80 are also called piezo-elements. To be specific, the first and second piezoelectric elements 60 and 80 of the present embodiment are brought into operation only after they are pressed from opposite sides, and do not detect an acceleration and the like only by the first and second piezoelectric elements 60 and 80 themselves. As an example, an element such as a piezoelectric transducer is of great convenience as it is capable of outputting an electric signal responsive, for example, to change in an acceleration if the piezoelectric transducer itself vibrates. However, the sensitivity of detection depends on the performance of the piezoelectric transducer, and therefore, there are limitations on the sensitivity.

In the present embodiment, the first and second piezoelectric elements 60 and 80 have a thickness of approximately 0.6 mm, and the transmission member 70 has the same length (thickness) of 5 mm as the support members 40. Accordingly, the clamping force variation detecting mechanism 50 is greater in length by 1.2 mm than the support members 40. The support members 40 maintain a constant distance of 5 mm between the substrate 30 and the pressure sensitive plate 10 as described above, so that a pre-load (clamping force) is applied to the clamping force variation detecting mechanism 50 in a stationary condition. By applying such a pre-load, the first and second piezoelectric elements 60 and 80 can detect variation in a clamping force that acts on the clamping force variation detecting mechanism 50 in analogous waveforms in the same phase with a high level of sensitivity.

Output powers from the first and second piezoelectric elements 60 and 80 are 0 V if there is no variation in a clamping force. Accordingly, the detecting module 100 of the present embodiment is not affected by differing weights of an organism to be measured. If a clamping force applied between the substrate 30 and the pressure sensitive plate 10 varies in response to minute vibration generated by the exhaled air, pulse and the like of the organism 1, the clamping force variation detecting mechanism 50 precisely detects this variation. In particular, the clamping force variation detecting mechanism 50 causes the first and second piezoelectric elements 60 and 80 to detect the same variation in a clamping force at the same time through the transmission member 70. This makes the first and second detected signals be substantially analogous signals having waveforms in the same phase. Piezoelectric properties make the first and second detected signals have waveforms corresponding to voltages to be output in response to strain stresses applied to the first and second piezoelectric element 60 and 80, respectively. To be specific, variation in a clamping force generated based on vibration acting on the pressure sensitive surface 12 is transmitted through the transmission member 70 to both the first and second piezoelectric elements 60 and 80 at the same time, and is converted to the substantially analogous first and second detected signals having waveforms in the same phase. Then, the signal combining unit 200 described later combines the first and second detected signals.

In particular, in the present embodiment, since the clamping force variation detecting mechanism 50 is arranged at the center of the pressure sensitive plate 10, the first and second piezoelectric elements 60 and 80 can simultaneously detect variation in pressure applied to the pressure sensitive plate 10 irrespective of the position of application of the pressure. Further, the clamping force variation detecting mechanism 50 is configured to detect variation in a clamping force acting between the pressure sensitive plate 10 and the substrate 30. Accordingly, even if a clamping force varies slowly at a nearly zero acceleration in response, for example, to body movement or breathing, the clamping force variation detecting mechanism 50 is capable of detecting this variation reliably. A conventional structure of directly detecting the vibration of the pressure sensitive plate 10 cannot detect this variation unless the pressure sensitive plate 10 itself vibrates sufficiently, or unless the pressure sensitive plate 10 itself is distorted significantly.

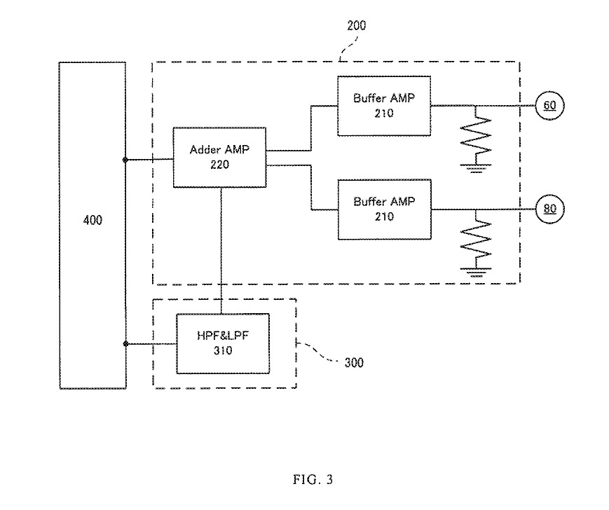

FIG. 3 shows a circuit structure for the signal combining unit 200 and the signal extracting unit 300. The signal combining unit 200 includes buffer amplifiers 210 provided to respective output cables of the first and second piezoelectric elements 60 and 80, and an adder circuit (adder amplifier) 220 for combining the first and second detected signals transmitted through the buffer amplifiers 210. An output signal from the signal combining unit 200 contains information about both breathing and heartbeat. Generally, breathing has a low frequency and heartbeat has a high frequency. Accordingly, the signal extracting unit 300 uses the output signal from the signal combining unit 200 to extract a high-frequency component by removing the frequency of breathing with a high-pass and low-pass filter 310. More specifically, the high-pass filter removes a low-frequency component generated by breathing itself, by body movement, and the like. Meanwhile, the low-pass filter removes noises to form a considerably high-frequency component. As a result, a breathing component and noises are removed to extract only a heartbeat component.

As described above, in the bioinformation detecting device 2 of the present embodiment, the components of variation in a clamping force simultaneously detected by the first and second piezoelectric elements 60 and 80 are combined by the signal combining unit 200. This allows significant increase of detection sensitivity. For example, a breathing rate is determined by detecting peaks in the smoothed waveform of an output signal from the signal combining unit 200. In some cases, detection with one piezoelectric element makes it impossible to detect peaks due to the influence of noises and the like. In contrast, peak values in a smoothed waveform can be made clear in the present embodiment, making it possible to enhance detection accuracy. As another example, for detection of heartbeat, an output signal from the signal extracting unit 300 is subjected to waveform processing such as first-order differentiation, and then negative peaks are detected in the waveform. If the negative peaks are detected with one piezoelectric element, the negative peaks have a wide range of variations, making it difficult to distinguish between a different noise component and the negative peaks. In contrast, the present embodiment forms homogenous negative peak levels and increases a difference between a different noise component and peak values. This makes it possible to make a determination of correctness to enhance detection accuracy.

Further, in the bioinformation detecting device 2 of the present embodiment, the presence of the substrate 30, the pressure sensitive plate 10, and the support members 40 applies a pre-load (clamping force) to the clamping force variation detecting mechanism 50 in a no-load condition to always maintain favorable detection sensitivity. This allows the detected signals of the first and second piezoelectric elements 60 and 80 to have the same phase. Accordingly, necessary information can be emphatically output easily by combining these detected signals as already described above. It is very unlikely that noise components generated depending on the respective circumstances of the first and second piezoelectric elements 60 and 80 will appear at the same time and will have the same phase. Thus, addition of the first and second detected signals still prevents a noise component from appearing emphatically, making it possible to enhance an S/N ratio.

### EXAMPLE AND COMPARATIVE EXAMPLE

Waveform characteristics actually detected by the bioinformation detecting device 2 according to the present embodiment will be described next. Waveform characteristics obtained by a bioinformation detecting device not publicly known are also described as Comparative Example. This bioinformation detecting device has substantially the same structure as the device 2, but includes one piezoelectric element in a clamping force variation detecting mechanism. Regarding test environment, a commercially available bed pad (of a thickness of 19 cm) was used as a bed, and the same person was laid on the bed pad in Example and Comparative Example to detect bioinformation under a head region and an abdominal region. In the waveform charts referred to below, a vertical axis indicates a voltage and a horizontal axis indicates time.

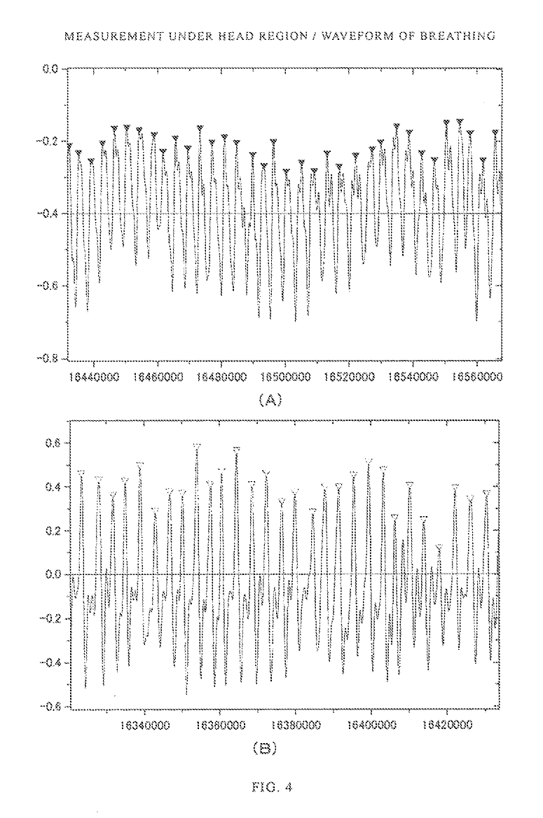

FIG. 4(A) shows a result obtained by smoothing a waveform (combined waveform of breathing and heartbeat) obtained from the output of the signal combining unit 200 when the bioinformation detecting device 2 of Example was placed under the head region to make a low-frequency component apparent, and thereafter determining the peak values of the low-frequency component. FIG. 4(B) shows a result obtained in Comparative Example under the same condition. These peak values indicate timing of breathing. As clearly seen from FIG. 4, a difference between the highest and lowest peak values in the waveform (A) of Example is such that the lowest peak value does not exceed a value that is about half the highest peak value. In contrast, a difference between the highest and lowest peak values is large in the waveform (B) of Comparative Example, making it difficult to distinguish between a breathing component and a noise component in some cases. In particular, Comparative Example experienced large amplitude variation, whereas Example experienced relatively small change of an amplitude itself.

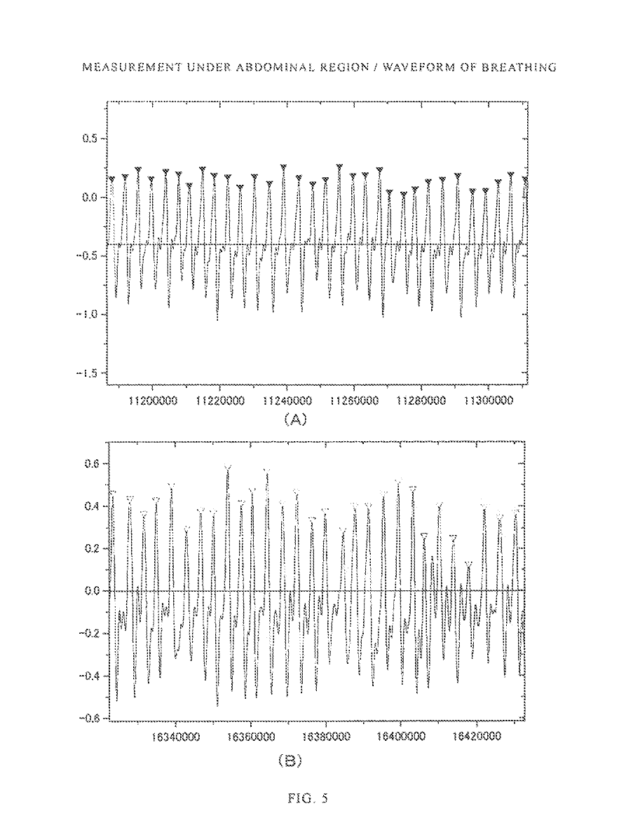

FIG. 5(A) shows a result obtained by smoothing a waveform (combined waveform of breathing and heartbeat) obtained from the output of the signal combining unit 200 when the bioinformation detecting device 2 of Example was placed under the abdominal region to make a low-frequency component apparent, and thereafter determining the peak values of the low-frequency component. FIG. 5(B) shows a result obtained in Comparative Example under the same condition. Like in FIG. 4, the waveform (A) of Example clearly shows that a difference between the highest and lowest peak values falls in a very small range. In contrast, a difference between the highest and lowest peak values is large in the waveform (B) of Comparative Example, making it difficult to distinguish between a breathing component and a noise component in some cases. In particular, Comparative Example experienced large amplitude variation, whereas Example experienced relatively small change of an amplitude itself.

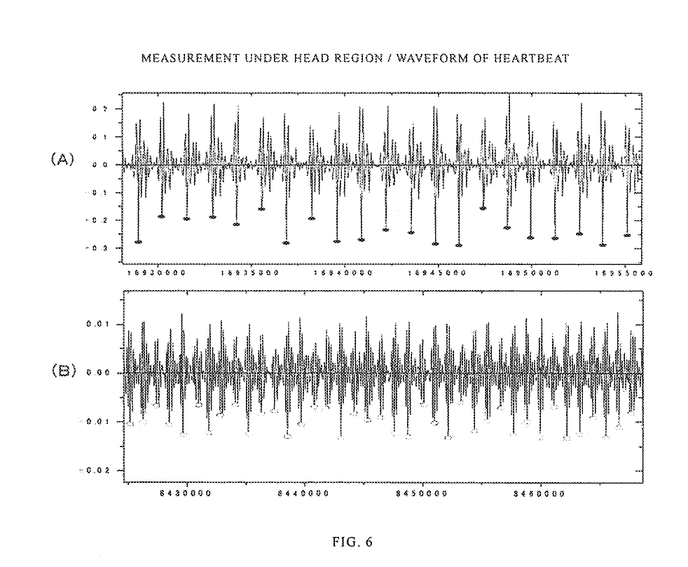

FIG. 6(A) shows a result obtained by utilizing a waveform (waveform of heartbeat) obtained from the output of the signal extracting unit 300 when the bioinformation detecting device 2 of Example was placed under the head region to determine the peak values of the waveform. FIG. 6(B) shows a result obtained in Comparative Example under the same condition. These peak values indicate timing of heartbeat. FIG. 6 clearly shows that a difference between the highest and lowest peak values in the waveform (A) of Example is such that the lowest peak value does not exceed a value that is about half the highest peak value, and that noises are reduced. In contrast, the waveform (B) of Comparative Example shows that a difference between the highest and lowest peak values is large and a noise component is relatively large, making it difficult to distinguish between a heartbeat component and a noise component in some cases.

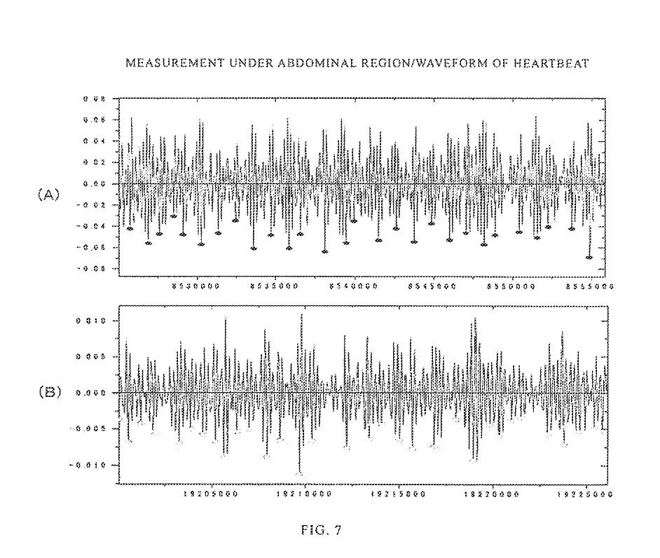

FIG. 7(A) shows a result obtained by utilizing a waveform (waveform of heartbeat) obtained from the output of the signal extracting unit 300 when the bioinformation detecting device 2 of Example was placed under the abdominal region to determine the peak values of the waveform. FIG. 6(B) shows a result obtained in Comparative Example under the same condition. Like FIG. 6, FIG. 7 also shows that a difference between the highest and lowest peak values in the waveform (A) of Example is such that the lowest peak value does not exceed a value that is about half the highest peak value. In contrast, the waveform (B) of Comparative Example shows that a difference between the highest and lowest peak values is large and a noise component is relatively large, making it difficult to distinguish between a heartbeat component and a noise component in some cases.

As understood from Example and Comparative Example, use of the bioinformation detecting device 2 including a clamping force variation detecting mechanism with two piezoelectric elements not only achieves amplification effect, but it also allows improvement of an S/N ratio by stabilizing peak values and reducing noises, leading to an increased degree of detection accuracy.

The bioinformation detecting device 1 of the present invention is not limited to the embodiment described above, but various changes may certainly be added to the bioinformation detecting device 1 without departing from the scope of the present invention.

### INDUSTRIAL APPLICABILITY

The bioinformation detecting device of the present invention using piezoelectric elements is applicable in a wide range of fields including detection and analysis of bioinformation such as breathing, medical checkup, and preventive medical care.

1. A bioinformation detecting device for detecting bioinformation such as a breathing condition, heartbeat, and body movement of an organism based on vibration generated by breathing motion of the organism, the bioinformation detecting device comprising:
a pressure sensitive plate with a pressure sensitive surface for detecting the vibration;
a substrate arranged to face the pressure sensitive plate;
a clamping force variation detecting mechanism arranged between the pressure sensitive plate and the substrate, the clamping force variation detecting mechanism detecting variation in a clamping force applied between the pressure sensitive plate and the substrate based on the vibration acting on the pressure sensitive surface; and
a signal combining unit for combining a plurality of detected signals output from the clamping force variation detecting mechanism, the bioinformation detecting device characterized in that
the clamping force variation detecting mechanism includes:
a first piezoelectric element arranged on the pressure sensitive plate on an opposite side of the pressure sensitive surface, the first piezoelectric element converting the variation in a clamping force acting on the first piezoelectric element itself to a first detected signal;
a transmission member arranged on the first piezoelectric element on an opposite side of the pressure sensitive plate, the transmission member transmitting the variation in a clamping force; and
a second piezoelectric element arranged between the transmission member and the substrate, the second piezoelectric element converting the variation in a clamping force acting on the second piezoelectric element itself to a second detected signal,
that the variation in a clamping force generated based on the vibration acting on the pressure sensitive surface is transmitted through the transmission member to both the first piezoelectric element and the second piezoelectric element, and is converted to the first and second detected signals, and that the first and second detected signals are combined by the signal combining unit, thereby detecting the bioinformation.

2. The bioinformation detecting device according to claim 1, characterized in that a support member for maintaining a constant distance between the pressure sensitive plate and the substrate is arranged between the pressure sensitive plate and the substrate in a region surrounding the clamping force variation detecting mechanism.

3. The bioinformation detecting device according to claim 2, characterized in that a pre-load is applied to the clamping force variation detecting mechanism in a stationary condition by setting a distance between the pressure sensitive plate and the substrate maintained by the support member to be smaller than a size of the clamping force variation detecting mechanism in a direction of the variation in a clamping force determined while no load is applied to the clamping force variation detecting mechanism.

4. The bioinformation detecting device according to claim 1, characterized by further comprising a signal extracting unit for extracting a heartbeat component by filtering an output signal from the signal combining unit.

5. The bioinformation detecting device according to claim 2, characterized in that the support member is provided at least in each of the four corners of the pressure sensitive plate, and
that the clamping force variation detecting mechanism is disposed at a position that is substantially the center of the pressure sensitive plate.
